(19) United States
(12) Patent Application Publication
(10) Pub. No.: US 2020/0041905 A1
(43) Pub. Date: Feb. 6, 2020

(54) RESIST UNDERLYING FILM-FORMING COMPOSITION CONTAINING AN AMIDE GROUP-CONTAINING POLYESTER

(71) Applicant: NISSAN CHEMICAL CORPORATION, Tokyo (JP)

(72) Inventors: Mamoru TAMURA, Toyama-shi (JP); Hiroto OGATA, Toyama-shi (JP); Yuki USUI, Toyama-shi (JP); Takahiro KISHIOKA, Toyama-shi (JP)

(73) Assignee: NISSAN CHEMICAL CORPORATION, Tokyo (JP)

(21) Appl. No.: 16/340,212

(22) PCT Filed: Oct. 3, 2017
(86) PCT No.: PCT/JP2017/035967

§ 371 Date: Apr. 8, 2019

(30) Foreign Application Priority Data
Oct. 14, 2016 (JP) 2016-202333

(51) Int. Cl. G03F 7/11; C07C 235/16; C08G 59/52; G03F 7/20

(52) U.S. Cl. CPC G03F 7/11; G03F 7/20; C08G 59/52; C07C 235/16

(57) ABSTRACT

A resist underlayer film-forming composition capable of providing a resist underlayer film exerting a sufficient anti-reflection function particularly in a KrF process, a high solvent resistance and a high dry etching speed, and enables the formation of a photoresist pattern having a good cross-sectional shape. The composition includes a copolymer containing: structural unit (A) derived from a diepoxy compound; and structural unit (B) derived from a compound represented by formula (1) [wherein: A represents a benzene or cyclohexane ring; X represents a hydrogen atom, alkyl or alkoxy group having 1 to 10 carbon atoms and optionally substituted by a halogen atom, or an alkoxycarbonyl group having 2 to 11 carbon atoms; and Y represents —COOH or -L-NHCO—Z—COOH (wherein: Z represents an alkylene group having 3 to 10 carbon atoms and optionally substituted by an oxygen atom, sulfur atom or nitrogen atom; and L represents a single bond or a spacer)].

### TECHNICAL FIELD

The present invention relates to a composition for forming a resist underlying film, which is to be formed between a substrate and a resist film (resist layer) formed thereon, and which is suitable for a lithography process in a method for the manufacture of semiconductors.

### BACKGROUND ART

When a resist film is subjected to exposure, reflected waves can adversely affect the resist film. A resist underlying film formed for the purpose of suppressing the adverse effect is called an antireflection film.

The resist underlying film is required to be easily formed merely by applying a resist underlying film-forming composition in the form of a solution and curing the composition. Therefore, the composition for forming the resist underlying film needs to contain a compound (polymer) which is readily cured by, for example, heating and to have a high solubility in a predetermined solvent.

Moreover, the resist underlying film is required to have a larger dry etching rate than that of a resist film formed on the film, i.e., to have a large selective ratio for dry etching rate.

Furthermore, it is desired that the resist pattern formed on the resist underlying film has a rectangular cross-section (straight bottom form free from the so-called undercut, footing and others), when taken along the direction perpendicular to the substrate. For example, the resist pattern having an undercut or footing profile would cause such problems that the resist pattern collapses or that a material to be processed (such as a substrate or an insulating film) cannot be processed into a desired form or size in the lithography process.

An antireflection film-forming composition comprising a polymer containing a sulfur atom in a predetermined amount is disclosed in Patent Literature 1 below. Further, a lithography antireflection film-forming composition comprising a reaction product obtained by a polyaddition reaction of an epoxy compound having two glycidyl groups with a nitrogen-containing aromatic compound having two thiol groups is disclosed in Patent Literature 2 below.

However, a composition used for forming an antireflection film exhibiting high performance particularly in photolithography using a KrF excimer laser has still been awaited.

### CITATION LIST

### Patent Literature

Patent Literature 1: WO 2005/088398 A1

Patent Literature 2: WO 2006/040918 A1

### SUMMARY OF INVENTION

### Technical Problem

Therefore, the technical problem underlying the present invention is to provide a composition which is used for forming an antireflection film for photolithography using a KrF excimer laser, and which satisfies the following properties.

(1) The resist underlying film formed from the composition has high antireflection effect and high resist pattern profile controlling ability.

(2) On the resist underlying film, a resist pattern having a good profile can be formed without causing intermixing with the resist film.

(3) The resist underlying film can be removed in a remarkably shorter time than the resist pattern under the conditions using CF₄ or an O₂/N₂ mixed gas as a dry etching gas.

Accordingly, an object of the present invention is to provide a composition for forming a resist underlying film which has a large selective ratio to the dry etching rate of the resist film, has a high solvent resistance, and exhibits a satisfactory k value at the wavelength (about 248 nm) of the KrF excimer laser. Another object is to provide a composition for forming a resist underlying film which gives the resist pattern formed on the resist underlying film in a desired profile.

### Solution to Problem

The present invention embraces the following.

[1] A compound represented by the following formula (1):

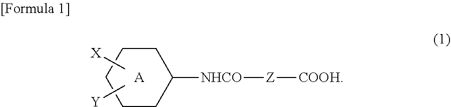

wherein:
A represents a benzene ring or a cyclohexane ring,
X represents a hydrogen atom, an optionally halo-substituted alkyl or alkoxy group having 1 to 10 carbon atoms, or an alkoxycarbonyl group having 2 to 11 carbon atoms,
Y represents —COOH or -L-NHCO—Z—COOH,
Z represents an alkylene group having 3 to 10 carbon atoms and being optionally interrupted by an oxygen atom, a sulfur atom, or a nitrogen atom, and
L represents a single bond or a spacer group,
with the proviso that the following compounds are excluded:

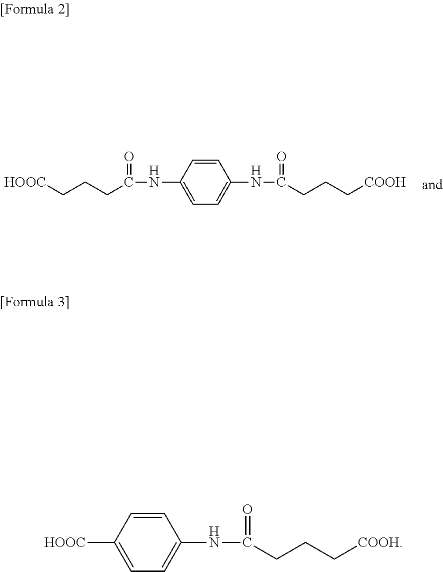 [SMILES: O=C(O)CCCC(=O)Nc1ccc(NC(=O)CCCC(=O)O)cc1 ; O=C(O)CCCC(=O)Nc1ccc(C(=O)O)cc1]

[2] The compound according to item [1], wherein A is a benzene ring.

[3] The compound according to item [1] or [2], wherein the spacer group -L- is represented by the following formula:

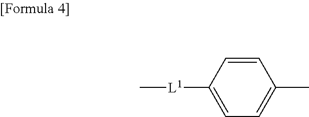

wherein L¹ represents a single bond, an oxygen atom, a carbonyl group, a sulfonyl group, or an optionally halo-substituted alkylene group having 1 to 6 carbon atoms.

[4] A copolymer comprising structural unit (A) derived from a diepoxy compound, and structural unit (B) derived from a compound represented by the following formula (1):

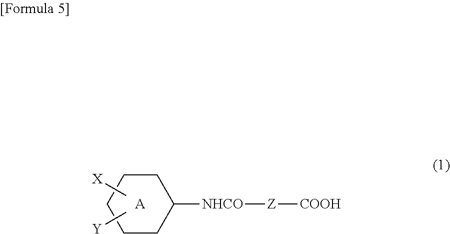

wherein:
A represents a benzene ring or a cyclohexane ring,
X represents a hydrogen atom, an optionally halo-substituted alkyl or alkoxy group having 1 to 10 carbon atoms, or an alkoxycarbonyl group having 2 to 11 carbon atoms,
Y represents —COOH or -L-NHCO—Z—COOH,
Z represents an alkylene group having 3 to 10 carbon atoms and being optionally interrupted by an oxygen atom, a sulfur atom, or a nitrogen atom, and
L represents a single bond or a spacer group.

[5] The copolymer according to item [4], wherein the diepoxy compound is represented by the following formula (2):

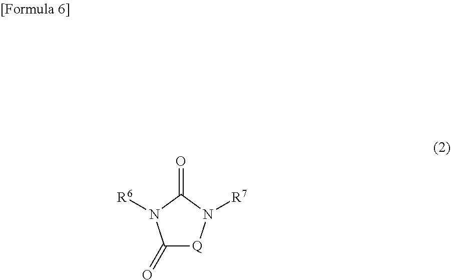

wherein:
R⁶ and R⁷ represent an epoxy-containing group, which may be the same or different, and
Q represents a group represented by the following formula (31), (32), or (33):

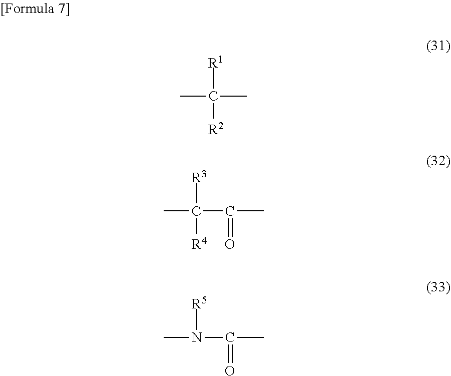

wherein each of R¹ to R⁴ independently represents a hydrogen atom, an alkyl group having 1 to 6 carbon atoms, an alkenyl group having 3 to 6 carbon atoms, a benzyl group, or a phenyl group, wherein the phenyl group is optionally substituted with at least one group selected from the group consisting of an alkyl group having 1 to 6 carbon atoms, a halogen atom, an alkoxy group having 1 to 6 carbon atoms, a nitro group, a cyano group, and an alkylthio group having 1 to 6 carbon atoms, and wherein R¹ and R², or R³ and R⁴ are optionally bonded to each other to form a ring having 3 to 6 carbon atoms, and
R⁵ represents a hydrogen atom, an alkyl group having 1 to 6 carbon atoms, an alkenyl group having 3 to 6 carbon atoms, an ether oxygen-interrupted alkyl group having 3 to 8 carbon atoms, a benzyl group, or a phenyl group.

[6] The copolymer according to item [4], wherein the diepoxy compound is represented by the following formula (4):

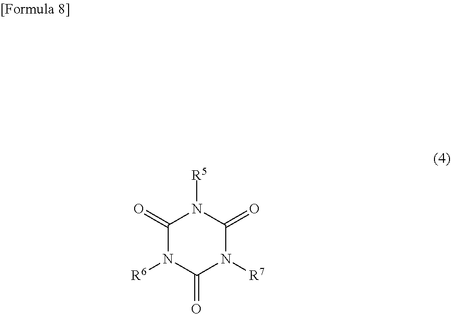

wherein:
R⁵ represents a hydrogen atom, an alkyl group having 1 to 6 carbon atoms, an alkenyl group having 3 to 6 carbon atoms, an ether oxygen-interrupted alkyl group having 3 to 8 carbon atoms, a benzyl group, or a phenyl group, and
R⁶ and R⁷ represent an epoxy-containing group.

[7] A resist underlying film-forming composition comprising a solvent and the copolymer according to any one of items [4] to [6].

[8] A resist underlying film-forming composition comprising a solvent and the copolymer according to any one of items [4] to [6], the composition giving a resist underlying film which absorbs a light having a wavelength of 248 nm.

[9] A method for forming a resist pattern for use in producing a semiconductor device, comprising the steps of: applying the resist underlying film-forming composition according to item [7] or [8] onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; and subjecting the exposed resist film to development.

[10] A resist underlying film comprising the copolymer according to any one of items [4] to [6], wherein the resist underlying film absorbs a light having a wavelength of 248 nm.

[11] A method for producing a semiconductor device, comprising the steps of: applying the resist underlying film-forming composition according to item [7] or [8] onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; subjecting the exposed resist film to development; and processing the semiconductor substrate using the resist film as a mask.

### Advantageous Effects of Invention

The resist underlying film formed from the resist underlying film-forming composition of the present invention has an absorption at 248 nm (KrF), which is indispensable to an antireflection film for photolithography using a KrF excimer laser, and exhibits satisfactory antireflection ability in a KrF process. Moreover, the resist underlying film has a high solvent resistance and a high dry etching rate. Furthermore, a photoresist pattern formed using the resist underlying film-forming composition of the present invention gives a cross-section profile having a good straight bottom form.

### DESCRIPTION OF EMBODIMENTS

The present invention provides a novel compound having at least one amide bond and two terminal carboxyl groups in the molecule, a copolymer having a structural unit derived from a diepoxy compound having two epoxy groups in the molecule and a structural unit derived from a compound having at least one amide bond and two terminal carboxyl groups in the molecule, a resist underlying film-forming composition comprising the above-mentioned copolymer, a resist underlying film comprising the copolymer, and a method for forming a resist pattern and a method for producing a semiconductor device, each using the above-mentioned resist underlying film-forming composition. They are consecutively described below.

1. Synthesis of the Copolymer

The copolymer may be produced by subjecting a compound represented by the following formula (1):

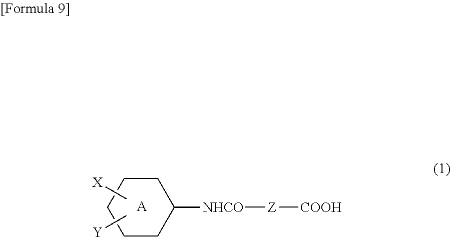

wherein:
A represents a benzene ring or a cyclohexane ring,
X represents a hydrogen atom, an optionally halo-substituted alkyl or alkoxy group having 1 to 10 carbon atoms, or an alkoxycarbonyl group having 2 to 11 carbon atoms,
Y represents —COOH or -L-NHCO—Z—COOH,
Z represents an alkylene group having 3 to 10 carbon atoms and being optionally interrupted by an oxygen atom, a sulfur atom, or a nitrogen atom, and
L represents a single bond or a spacer group
   and an appropriate diepoxy compound to copolymerization in a conventional manner.

In the present invention, the term “copolymer” refers to a copolymer which is not necessarily limited to a compound of high molecular weight, and therefore it excludes a monomer but includes an oligomer.

The compound represented by formula (1) above and the diepoxy compound may be used each alone or in combination of two or more.

### 1.1. Monomer

### 1.1.1. Compound Represented by Formula (1)

The compound represented by formula (1) above is a compound having at least one amide bond and two terminal carboxyl groups in the molecule.

In formula (1), A represents a benzene ring or a cyclohexane ring, preferably a benzene ring. X represents a hydrogen atom, an optionally halo-substituted alkyl or alkoxy group having 1 to 10 carbon atoms, or an alkoxycarbonyl group having 2 to 11 carbon atoms.

In the present invention, the “alkyl group” includes a linear, branched, and cyclic alkyl group. Examples of alkyl groups having 1 to 10 carbon atoms include a methyl group, an ethyl group, a n-propyl group, an i-propyl group, a cyclopropyl group, a n-butyl group, an i-butyl group, a s-butyl group, a t-butyl group, a cyclobutyl group, a 1-methyl-cyclopropyl group, a 2-methyl-cyclopropyl group, a n-pentyl group, a 1-methyl-n-butyl group, a 2-methyl-n-butyl group, a 3-methyl-n-butyl group, a 1,1-dimethyl-n-propyl group, a 1,2-dimethyl-n-propyl group, a 2,2-dimethyl-n-propyl group, a 1-ethyl-n-propyl group, a 1,1-diethyl-n-propyl group, a cyclopentyl group, a 1-methyl-cyclobutyl group, a 2-methyl-cyclobutyl group, a 3-methyl-cyclobutyl group, a 1,2-dimethyl-cyclopropyl group, a 2,3-dimethyl-cyclopropyl group, a 1-ethyl-cyclopropyl group, a 2-ethyl-cyclopropyl group, a n-hexyl group, a 1-methyl-n-hexyl group, a 1-methyl-n-pentyl group, a 2-methyl-n-pentyl group, a 3-methyl-n-pentyl group, a 4-methyl-n-pentyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, a 1-ethyl-n-butyl group, a 2-ethyl-n-butyl group, a 1,1,2-trimethyl-n-propyl group, a 1,2,2-trimethyl-n-propyl group, a 1-ethyl-1-methyl-n-propyl group, a 1-ethyl-2-methyl-n-propyl group, a cyclohexyl group, a 1-methyl-cyclopentyl group, a 2-methyl-cyclopentyl group, a 3-methyl-cyclopentyl group, a 1-ethyl-cyclobutyl group, a 2-ethyl-cyclobutyl group, a 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a 1-n-propyl-cyclopropyl group, a 2-n-propyl-cyclopropyl group, a 1-i-propyl-cyclopropyl group, a 2-i-propyl-cyclopropyl group, a 1,2,2-trimethyl-cyclopropyl group, a 1,2,3-trimethyl-cyclopropyl group, a 2,2,3-trimethyl-cyclopropyl group, a 1-ethyl-2-methyl-cyclopropyl group, a 2-ethyl-1-methyl-cyclopropyl group, a 2-ethyl-2-methyl-cyclopropyl group, a 2-ethyl-3-methyl-cyclopropyl group, a n-heptyl group, a 1-methyl-n-heptyl group, a n-octyl group, a 1-methyl-n-octyl group, a n-nonyl group, a 1-methyl-n-nonyl group, and a n-decanyl group.

Preferred are alkyl groups having 1 to 8 carbon atoms, more preferred are alkyl groups having 1 to 6 carbon atoms, and most preferred are a methyl group, an ethyl group, a n-propyl group, an i-propyl group, and a cyclopropyl group.

Examples of an alkoxy group having 1 to 10 carbon atoms include the above-mentioned alkyl group having an ether oxygen atom (—O—) bonded to the carbon atom at the terminal. The structure of the alkoxy group is preferably linear or branched. The number of carbon atoms of the alkoxy group is preferably 1 to 8, more preferably 1 to 6, most preferably 1 to 3. Examples of such alkoxy groups include a methoxy group, an ethoxy group, a n-propoxy group, an isopropoxy group, a n-butoxy group, an isobutoxy group, a sec-butoxy group, a tert-butoxy group, a n-pentyloxy group, and a n-hexyloxy group.

Examples of an alkoxycarbonyl group having 2 to 11 carbon atoms include the above-mentioned alkoxy group having a carbonyl group (—CO—) bonded to the carbon atom at the terminal. The structure of the alkoxycarbonyl group is preferably linear or branched. The number of carbon atoms of the alkoxycarbonyl group is preferably 2 to 11, more preferably 2 to 7, most preferably 2 to 4. Examples of such alkoxycarbonyl groups include a methoxycarbonyl group, an ethoxycarbonyl group, a n-propoxycarbonyl group, an isopropoxycarbonyl group, a n-butoxycarbonyl group, an isobutoxycarbonyl group, a sec-butoxycarbonyl group, a tert-butoxycarbonyl group, a n-pentyloxycarbonyl group, and a n-hexyloxycarbonyl group.

With respect to the “halogen”, preferred are a fluorine atom, a chlorine atom, a bromine atom, and an iodine atom; and especially preferred are, for example, a fluorine atom and a chlorine atom.

In formula (1), Y represents —COOH or -L-NHCO—Z—COOH, Z represents an alkylene group having 3 to 10 carbon atoms, preferably having 3 to 6 carbon atoms, and being optionally interrupted by an oxygen atom, a sulfur atom, or a nitrogen atom, preferably by an oxygen atom, and L represents a single bond or a spacer group.

The spacer group is preferably represented by the following formula:

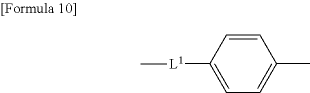

wherein L¹ represents a single bond, an oxygen atom, a carbonyl group, a sulfonyl group, or an optionally halo-substituted alkylene group having 1 to 6 carbon atoms, preferably having 1 to 3 carbon atoms.
   L¹ is preferably a sulfonyl group.

The compound represented by formula (1) may be obtained by reacting a diamine having a desired structure and an acid anhydride in a conventional manner. Specific examples of the compounds are shown in the Synthesis Examples below.

### 1.1.2. Compound Represented by Formula (2)

The compound represented by formula (2) is a diepoxy compound having two epoxy groups in the molecule.

The diepoxy compound preferably has the following formula (2):

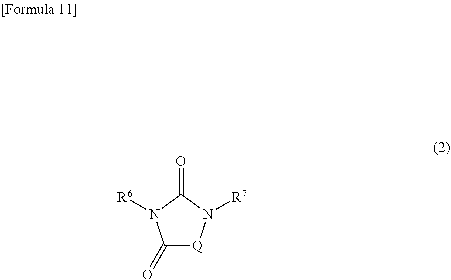

wherein:
R⁶ and R⁷ represent an epoxy-containing group, which may be the same or different, and
Q represents a group represented by the following formula (31), (32), or (33):

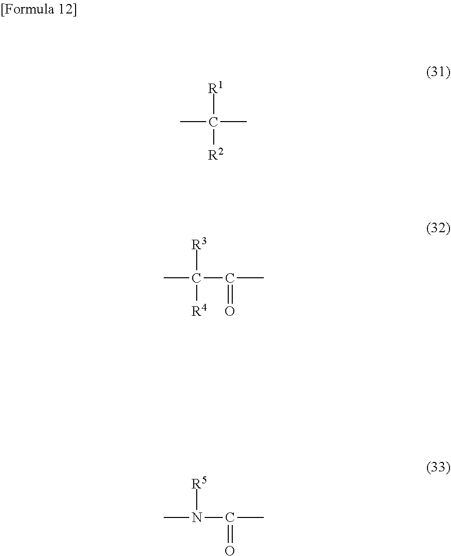

wherein each of R¹ to R⁴ independently represents a hydrogen atom, an alkyl group having 1 to 6 carbon atoms, an alkenyl group having 3 to 6 carbon atoms, a benzyl group, or a phenyl group, wherein the phenyl group is optionally substituted with at least one group selected from the group consisting of an alkyl group having 1 to 6 carbon atoms, a halogen atom, an alkoxy group having 1 to 6 carbon atoms, a nitro group, a cyano group, and an alkylthio group having 1 to 6 carbon atoms, and wherein R¹ and R², or R³ and R⁴ are optionally bonded to each other to form a ring having 3 to 6 carbon atoms, and
R⁵ represents a hydrogen atom, an alkyl group having 1 to 6 carbon atoms, an alkenyl group having 3 to 6 carbon atoms, an ether oxygen-interrupted alkyl group having 3 to 8 carbon atoms, a benzyl group, or a phenyl group.

The diepoxy compound more preferably has the following formula (4):

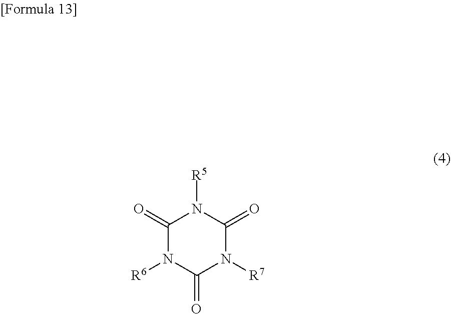

wherein:
R⁵ represents a hydrogen atom, an alkyl group having 1 to 6 carbon atoms, an alkenyl group having 3 to 6 carbon atoms, an ether oxygen-interrupted alkyl group having 3 to 8 carbon atoms, a benzyl group, or a phenyl group, and
R⁶ and R⁷ represent an epoxy-containing group.

The epoxy-containing group refers to a group having oxacyclopropane (oxirane), which is an ether of a three-membered ring, in the structural formula.

For example, the epoxy-containing group is a group represented by the following formula:

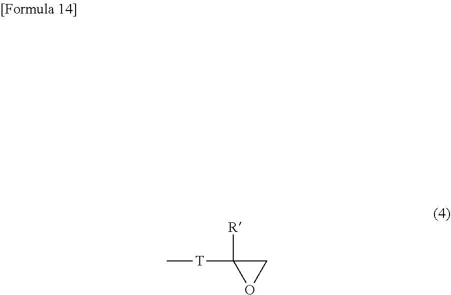

wherein T represents a single bond or a group represented by the formula: -Q′-X′—, and R′ represents a hydrogen atom, or a linear or branched alkyl group optionally interrupted by an oxygen atom, and R′ may bond to a carbon atom adjacent to the carbon atom to which R′ is bonded to form a ring.

In the formula above, Q′ represents alkylene having 1 to 10 carbon atoms, preferably having 1 to 6 carbon atoms, and is optionally mono- or multi-substituted with F, Cl, Br, I, or CN, wherein one or more CH₂ groups which are not adjacent to each other are optionally independently replaced by —O—, —S—, —NH—, —NR⁰—, —SiR⁰R⁰⁰—, —CO—, —COO—, —OCO—, —OCO—O—, —S—CO—, —CO—S—, —NR⁰—CO—O—, —O—CO—NR⁰—, —NR⁰—CO—NR⁰—, —CH═CH—, or —C≡C— so that an oxygen atom(s) and/or a sulfur atom(s) are not directly bonded to each other, and

X′ represents —O—, —S—, —CO—, —COO—, —OCO—, —O—COO—, —CO—NR⁰—, —NR⁰—CO—, —NR⁰—CO—NR⁰—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CR⁰—, —CY²═CY³—, —C≡C—, —CH═CH—COO—, —OCO—CH═CH—, or a single bond,

wherein each of R⁰ and R⁰⁰ independently represents H or alkyl having 1 to 10 carbon atoms, and

each of Y² and Y³ independently represents H, F, Cl, or CN.

X′ is preferably —O—, —S—, —CO—, —COO—, —OCO—, —O—COO—, —CO—NR⁰—, —NR⁰—CO—, —NR⁰—CO—NR⁰—, or a single bond.

Q′ is typically, for example, —(CH₂)ₚ₁—, —(CH₂CH₂O)_{q1}—CH₂CH₂—, —CH₂CH₂—S—CH₂CH₂—, —CH₂CH₂—NH—CH₂CH₂—, or —(SiR⁰R⁰⁰—O)ₚ₁—, wherein p1 is an integer of 1 to 10, q1 is an integer of 1 to 3, and R⁰ and R⁰⁰ are as defined above.

Examples of especially preferred group —X′-Q′- include —(CH₂)ₚ₁—, —O—(CH₂)ₚ₁—, —OCO—(CH₂)ₚ₁—, and —OCOO—(CH₂)ₚ₁—.

Examples of especially preferred group Q′ include ethylene, propylene, butylene, pentylene, hexylene, heptylene, octylene, nonylene, decylene, ethyleneoxyethylene, methyleneoxybutylene, ethylenethioethylene, ethylene-N-methyliminoethylene, ethenylene, propenylene, and butenylene, all of which are linear.

Examples of linear or branched alkyl group having 1 to 6 carbon atoms include a methyl group, an ethyl group, a n-propyl group, an i-propyl group, a n-butyl group, an i-butyl group, a s-butyl group, a t-butyl group, a n-pentyl group, a 1-methyl-n-butyl group, a 2-methyl-n-butyl group, a 3-methyl-n-butyl group, a 1,1-dimethyl-n-propyl group, a 1,2-dimethyl-n-propyl group, a 2,2-dimethyl-n-propyl group, a 1-ethyl-n-propyl group, a 1,1-diethyl-n-propyl group, a n-hexyl group, a 1-methyl-n-hexyl group, a 1-methyl-n-pentyl group, a 2-methyl-n-pentyl group, a 3-methyl-n-pentyl group, a 4-methyl-n-pentyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, a 1-ethyl-n-butyl group, a 2-ethyl-n-butyl group, a 1,1,2-trimethyl-n-propyl group, a 1,2,2-trimethyl-n-propyl group, a 1-ethyl-1-methyl-n-propyl group, and a 1-ethyl-2-methyl-n-propyl group. Preferred are a methyl group, an ethyl group, a n-propyl group, and an i-propyl group.

Examples of cyclic alkyl group include a cyclopropyl group, a cyclobutyl group, a 1-methyl-cyclopropyl group, a 2-methyl-cyclopropyl group, a cyclopentyl group, a 1-methyl-cyclobutyl group, a 2-methyl-cyclobutyl group, a 3-methyl-cyclobutyl group, a 1,2-dimethyl-cyclopropyl group, a 2,3-dimethyl-cyclopropyl group, a 1-ethyl-cyclopropyl group, a 2-ethyl-cyclopropyl group, a cyclohexyl group, a 1-methyl-cyclopentyl group, a 2-methyl-cyclopentyl group, a 3-methyl-cyclopentyl group, a 1-ethyl-cyclobutyl group, a 2-ethyl-cyclobutyl group, a 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a 1-n-propyl-cyclopropyl group, a 2-n-propyl-cyclopropyl group, a 1-i-propyl-cyclopropyl group, a 2-i-propyl-cyclopropyl group, a 1,2,2-trimethyl-cyclopropyl group, a 1,2,3-trimethyl-cyclopropyl group, a 2,2,3-trimethyl-cyclopropyl group, a 1-ethyl-2-methyl-cyclopropyl group, a 2-ethyl-1-methyl-cyclopropyl group, a 2-ethyl-2-methyl-cyclopropyl group, and a 2-ethyl-3-methyl-cyclopropyl group. Preferred is a cyclopropyl group.

Examples of alkenyl group having 3 to 6 carbon atoms include a propenyl group, a butenyl group, a pentenyl group, a cyclopentenyl group, a hexenyl group, and a cyclohexenyl group.

Examples of alkoxy group having 1 to 6 carbon atoms include a methoxy group, an ethoxy group, a n-propoxy group, an i-propoxy group, a n-butoxy group, an i-butoxy group, a s-butoxy group, a t-butoxy group, a 2-methylbutoxy group, a n-pentoxy group, and a n-hexoxy group.

Examples of alkylthio group having 1 to 6 carbon atoms include a methylthio group, an ethylthio group, a n-propylthio group, an i-propylthio group, a n-butylthio group, an i-butylthio group, a s-butylthio group, a t-butylthio group, a 2-methylbutylthio group, a n-pentylthio group, and a n-hexylthio group.

Examples of ether oxygen-interrupted alkyl group having 3 to 8 carbon atoms include a 2-methoxyethyl group.

Examples of diepoxy compound are not limited to those mentioned below, but, for example include 1,4-butanediol diglycidyl ether, 1,2-epoxy-4-(epoxyethyl)cyclohexane, diethylene glycol diglycidyl ether, diglycidyl 1,2-cyclohexanedicarboxylate, 4,4′-methylenebis(N,N-diglycidylaniline), 3,4-epoxycyclohexylmethyl-3,4-epoxycyclohexanecarboxylate, bisphenol-A-diglycidyl ether, bisphenol-S-diglycidyl ether, resorcinol diglycidyl ether, diglycidyl phthalate, neopentyl glycol diglycidyl ether, polypropylene glycol diglycidyl ether, tetrabromobisphenol-A-diglycidyl ether, bisphenol hexafluoroacetone diglycidyl ether, pentaerythritol diglycidyl ether, monoallyldiglycidyl isocyanurate, 1,4-bis(2,3-epoxypropoxyperfluoroisopropyl)cyclohexane, resorcin diglycidyl ether, 1,6-hexanediol diglycidyl ether, polyethylene glycol diglycidyl ether, phenylglycidyl ether, p-tertiarybutylphenylglycidyl ether, adipic acid diglycidyl ether, o-phthalic acid diglycidyl ether, 1,2,7,8-diepoxyoctane, 1,6-dimethylolperfluorohexane diglycidyl ether, 4,4′-bis(2,3-epoxypropoxyperfluoroisopropyl)diphenyl ether, 2,2-bis(4-glycidyloxyphenyl)propane, 3,4-epoxycyclohexylmethyl-3′,4′-epoxycyclohexanecarboxylate, 2-(3,4-epoxycyclohexyl)-3′,4′-epoxy-1,3-dioxane-5-spirocyclohexane, 1,2-ethylenedioxy-bis(3,4-epoxycyclohexylmethane), 4′,5′-epoxy-2′-methylcyclohexylmethyl-4,5-epoxy-2-methylcyclohexanecarboxylate, ethylene glycol-bis(3,4-epoxycyclohexanecarboxylate), bis-(3,4-epoxycyclohexylmethyl) adipate, and bis(2,3-epoxycyclopentyl) ether.

The above diepoxy compounds may be produced in a conventional manner, and, for example, may be produced from a compound having two or more hydroxyl groups or carboxyl groups, such as a diol compound, a triol compound, a dicarboxylic acid compound, or a tricarboxylic acid compound, and a glycidyl compound, such as epichlorohydrin. Alternatively, the diepoxy compounds are commercially available.

### 1.2. Catalyst

The reaction of a compound represented by formula (1) with a compound represented by formula (2) may be accelerated using an appropriate catalyst. Such a catalyst is a catalyst that activates an epoxy group. Examples of a catalyst that activates an epoxy group include quaternary phosphonium salts, such as ethyltriphenylphosphonium bromide, and quaternary ammonium salts, such as benzyltriethylammonium chloride. The amount of the catalyst used may be appropriately selected, but an appropriate amount of the catalyst used may be selected from, for example, an amount in the range of from 0.1 to 10% by mass, based on the total mass of the compound represented by formula (1) and the compound represented by formula (2) above, which are raw material monomers.

### 1.3. Solvent

The reaction of a compound represented by formula (1) with a compound represented by formula (2) may be accelerated using an appropriate solvent. The type and amount of the solvent used may be appropriately selected. Examples of the solvent include ethoxyethanol, methoxyethanol, 1-methoxy-2-propanol, propylene glycol monomethyl ether, dioxane, N,N-2-trimethylpropionamide, and cyclohexanone.

### 1.4. Reaction Conditions

One or two or more types of the compound represented by formula (1) above and one or two or more types of the compound represented by formula (2) above are dissolved in an appropriate solvent in an appropriate molar ratio, and subjected to copolymerization in the presence of a catalyst that activates an epoxy group.

There is no particular limitation to the molar ratio of the compound represented by formula (1) above and the compound represented by formula (2) above which are charged for the reaction; however, generally, the molar ratio of formula (1):formula (2) is in the range of from 85:115 to 115:85, preferably from 90:110 to 110:90.

The temperature and time for the polymerization reaction may be appropriately selected, but the temperature is preferably in the range of from 80 to 160° C., and the time is preferably in the range of from 2 to 50 hours.

### 1.5. Copolymer

Examples of specific structure of the copolymer comprising structural unit (A) derived from a diepoxy compound and structural unit (B) derived from a compound represented by formula (1) are shown in the below-described Synthesis Examples.

The weight average molecular weight of the copolymer, as determined by a GPC (gel permeation chromatography) method, varies depending on, for example, the application solvent used and the solution viscosity, but is, for example, in the range of from 1,000 to 50,000, preferably from 2,000 to 20,000, in terms of a molecular weight determined using a conversion calibration curve obtained from polystyrene.

2. Preparation of the Composition

An additive is added to the copolymer obtained as described above, and the resultant mixture is dissolved in an appropriate solvent, to obtain the resist underlying film-forming composition of the present invention.

### 2.1. Copolymer Component

The copolymer may be isolated from the above-obtained copolymer solution and then used in the preparation of the resist underlying film-forming composition, but the above-obtained copolymer solution may be used as such in the resist underlying film-forming composition.

### 2.2. Additives

The resist underlying film-forming composition of the present invention may further contain a crosslinkable compound and a sulfonic acid compound. The proportion of the sulfonic acid compound to the copolymer contained in the resist underlying film-forming composition of the present invention is not particularly limited, but is, for example, not less than 0.1% by mass and not more than 13% by mass, preferably not less than 0.5% by mass and not more than 5% by mass. The crosslinkable compound is called also as a crosslinking agent, and is, for example, a nitrogen-containing compound having 2 to 4 nitrogen atoms and being substituted with a methylol group or an alkoxymethyl group. The proportion of the crosslinkable compound to the copolymer contained in the resist underlying film-forming composition of the present invention is not particularly limited, but is, for example, in the range of from 5 to 50% by mass.

Preferred specific examples of the sulfonic acid compound include p-toluenesulfonic acid, 4-hydroxybenzenesulfonic acid, trifluoromethanesulfonic acid, pyridinium p-toluenesulfonate, pyridinium 4-hydroxybenzenesulfonate, camphorsulfonic acid, 5-sulfosalicylic acid, 4-chlorobenzenesulfonic acid, 4-hydroxybenzenesulfonic acid, benzenedisulfonic acid, 1-naphthalenesulfonic acid, and pyridinium 1-naphthalenesulfonate.

Preferred specific examples of the crosslinkable compound (crosslinking agent) include hexamethoxymethylmelamine, tetramethoxymethylglycoluril, tetramethoxymethylbenzoguanamine, 1,3,4,6-tetrakis(methoxymethyl)glycoluril, 1,3,4,6-tetrakis(butoxymethyl)glycoluril, 1,3,4,6-tetrakis(hydroxymethyl)glycoluril, 1,3-bis(hydroxymethyl)urea, 1,1,3,3-tetrakis(butoxymethyl)urea, and 1,1,3,3-tetrakis(methoxymethyl)urea, and more preferred is tetramethoxymethylglycoluril.

The sulfonic acid compound is an additive which functions as a crosslinking accelerator, and which, for example, like 4-hydroxybenzenesulfonic acid (also called p-phenolsulfonic acid), contributes to suppress the occurrence of footing of the resist pattern cross-section, when taken along the direction perpendicular to the substrate, to achieve a desired cross-section form (substantially rectangular form).

The resist underlying film-forming composition of the present invention may contain a phenol derivative. The phenol derivative is an additive which, like 4-hydroxybenzenesulfonic acid, contributes to suppress the occurrence of footing of the resist pattern cross-section, when taken along the direction perpendicular to the substrate, to achieve a desired cross-section form (substantially rectangular form). Specific examples of phenol derivative include 4-methylsulfonylphenol, bisphenol S, bisphenol AF, 4-cyanophenol, 3,4,5-trifluorophenol, 4-hydroxybenzotrifluoride, 2,3,5,6-tetrafluoro-4-(trifluoromethyl)phenol, and 2,6-dichloro-4-(methylsulfonyl)phenol. The proportion of the phenol derivative to the copolymer contained in the resist underlying film-forming composition of the present invention is not particularly limited, but is, for example, in the range of from 0.1 to 20% by mass.

The resist underlying film-forming composition of the present invention may contain a surfactant. The surfactant is an additive for improving the application properties of the composition to a substrate. A known surfactant, such as a nonionic surfactant or a fluorine surfactant, may be used, and may be added in an amount of, for example, 0.1 to 5% by mass, based on the mass of the copolymer contained in the resist underlying film-forming composition of the present invention.

When the component left behind the removal of the solvent from the resist underlying film-forming composition of the present invention is defined as solid component, the solid component includes the copolymer and the above-mentioned various additives added occasionally.

The concentration of the solid component in the resist underlying film-forming composition is, for example, in the range of from 0.1 to 15% by mass, preferably from 0.1 to 10% by mass.

### 2.3. Solvent

Specific examples of solvent contained in the resist underlying film-forming composition of the present invention include propylene glycol monomethyl ether (PGME), propylene glycol monomethyl ether acetate (PGMEA), propylene glycol monopropyl ether, methyl ethyl ketone, ethyl lactate, cyclohexanone, N,N-2-trimethylpropionamide, γ-butyrolactone, N-methylpyrrolidone, methyl 2-hydroxyisobutyrate, ethyl 3-ethoxypropionate, and mixtures of two or more solvents selected from the above solvents. The solvent used at the time of preparation of the copolymer may be contained as such in the composition.

The proportion of the solvent to the resist underlying film-forming composition of the present invention is not particularly limited, but is, for example, not less than 90% by mass and not more than 99.9% by mass.

3. Method for Forming Resist Pattern

The resist underlying film-forming composition of the present invention may be applied to a lithography process in a method for the manufacture of a semiconductor device. The lithography process comprises the steps of: applying the resist underlying film-forming composition of the present invention onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; and subjecting the exposed resist film to development; thus, the lithography process permits formation of a resist pattern on the resist underlying film.

### 3.1. Formation of Resist Underlying Film

### 3.1.1. Substrate

The semiconductor substrate is represented by a silicon wafer, but an SOI (silicon on insulator) substrate, or a compound semiconductor wafer, such as gallium arsenide (GaAs), indium phosphide (InP), or gallium phosphide (GaP), may be used. A semiconductor substrate having formed thereon an insulating film, such as a silicon oxide film, a nitrogen-containing silicon oxide film (SiON film), or a carbon-containing silicon oxide film (SiOC film), may be used, and, in such a case, the resist underlying film-forming composition of the present invention is applied onto the insulating film.

### 3.1.2. Application

The application of the resist underlying film-forming composition of the present invention may be conducted in a conventional manner, and the composition may be applied by an appropriate application method, for example, using a spinner or a coater.

### 3.1.3. Baking

The obtained film applied is baked to form a resist underlying film. The baking conditions are appropriately selected from those at a baking temperature of 80 to 500° C., or 80 to 250° C. for a baking time of 0.3 to 60 minutes. Preferred are the conditions at a baking temperature of 100 to 500° C. for a baking time of 0.5 to 2 minutes. The resist underlying film formed has a thickness of, for example, 10 to 1,000 nm, or 20 to 500 nm, or 50 to 300 nm, or 100 to 200 nm, or 10 to 100 nm.

### 3.2. Formation of Resist Film

On the above-obtained resist underlying film, for example, a photoresist film is formed. The formation of the photoresist film may be carried out by a known method, specifically by applying a photoresist composition solution onto the resist underlying film and baking the applied composition.

In the present invention, an inorganic lower-layer film and an organic lower-layer film are formed on a substrate, and then the resist underlying film of the present invention is formed further thereon, and a photoresist film may further be formed on the resist underlying film. By virtue of this, even when the pattern width of the photoresist film is narrowed for pattern microfabrication and the thickness of the photoresist film applied is reduced for preventing pattern collapse, processing of the substrate becomes possible by selecting an appropriate etching gas. For example, it may be possible to process the resist underlying film of the present invention with a fluorine-based gas having a satisfactorily fast etching rate for the photoresist as an etching gas, it may be possible to process the organic lower-layer film with an oxygen-based gas having a satisfactorily fast etching rate for the resist underlying film of the present invention as an etching gas, and it may be possible to process the substrate with a fluorine-based gas having a satisfactorily fast etching rate for the organic lower-layer film as an etching gas.

There is no particular limitation to the photoresist for the film formed on the resist underlying film of the present invention, as long as it is sensitive to a light used in the exposure. Any of a negative photoresist and a positive photoresist may be used. They include, for example, a positive photoresist comprising a novolak resin and a 1,2-naphthoquinonediazidosulfonate; a chemical amplification photoresist comprising a photo-acid generator and a binder having a group which is decomposed by an acid to increase the alkali solubility; a chemical amplification photoresist comprising an alkali-soluble binder and a low-molecular weight compound which is decomposed by an acid to increase the alkali solubility of the photoresist; and a chemical amplification photoresist comprising a photo-acid generator, a binder having a group which is decomposed by an acid to increase the alkali solubility, and a low-molecular weight compound which is decomposed by an acid to increase the alkali solubility of the photoresist. For example, they include trade name APEX-E, manufactured by Shipley Company, Inc., trade name PAR710, manufactured by Sumitomo Chemical Co., Ltd., and trade name SEPR430, manufactured by Shin-Etsu Chemical Co., Ltd. They also include fluorine atom-containing polymer photoresists disclosed in, for example, Proc. SPIE, Vol. 3999, 330-334 (2000), Proc. SPIE, Vol. 3999, 357-364 (2000), and Proc. SPIE, Vol. 3999, 365-374 (2000).

### 3.4. Exposure

Next, exposure through a predetermined mask is conducted. For utilizing the advantages of the present invention, in the exposure, a KrF excimer laser (wavelength: 248 nm) is preferably used as a light source. An ArF excimer laser (wavelength: 193 nm), an EUV (wavelength: 13.5 nm) light, or an electron beam may be used instead of a KrF excimer laser. The “EUV” is an abbreviation for extreme ultraviolet. The resist for forming a resist film may be either positive or negative. In the present invention, a KrF excimer laser is suitably used, but a chemical amplification resist sensitive to ArF, an EUV light, or an electron beam may be used. After the exposure, if necessary, post exposure bake may be carried out. The post exposure bake is conducted under the conditions appropriately selected from those at a baking temperature of 70 to 150° C. for a baking time of 0.3 to 10 minutes.

### 3.5. Development

Next, development using a developer is conducted. In the development, when a positive photoresist is used, for example, the exposed portion of the photoresist film is removed, to form a photoresist pattern.

Examples of developer include alkaline aqueous solutions, e.g., aqueous solutions of an alkali metal hydroxide, such as potassium hydroxide or sodium hydroxide, aqueous solutions of a quaternary ammonium hydroxide, such as tetramethylammonium hydroxide, tetraethylammonium hydroxide, or choline, and aqueous solutions of an amine, such as ethanolamine, propylamine, or ethylenediamine. Moreover, for example, a surfactant may be added to these developers. The conditions for the development are appropriately selected from those at a temperature of 5 to 50° C. for a time of 10 to 600 seconds.

4. Production of Semiconductor Device

A part of the resist underlying film of the present invention (intermediate layer) is removed and patterned using the photoresist film (upper layer) having the pattern formed as described above as a protective film. And then, the semiconductor substrate is processed using the film comprising the patterned photoresist film (upper layer) and the resist underlying film of the present invention (intermediate layer) as a protective film.

Alternatively, a part of the resist underlying film of the present invention (intermediate layer) is removed and patterned using the photoresist film (upper layer) having the pattern formed as described above as a protective film. And then, a part of the inorganic lower-layer film (lower layer) is removed and patterned using the film comprising the patterned photoresist film (upper layer) and the resist underlying film of the present invention (intermediate layer) as a protective film. Finally, the semiconductor substrate is processed using the patterned resist underlying film of the present invention (intermediate layer) and the inorganic lower-layer film (lower layer) as a protective film.

It may also possible to further form an organic lower-layer film (such as an amorphous carbon film, an organic hard mask, or a spin-on carbon film) under the inorganic lower-layer film (lower layer), and process the semiconductor substrate.

After the photoresist film is patterned, the resist underlying film of the present invention (intermediate layer) is first removed by dry etching at the portion from which the photoresist film has been removed, so as to expose the inorganic lower-layer film (lower layer). For the dry etching of the resist underlying film of the present invention, such a gas as tetrafluoromethane (CF₄), perfluorocyclobutane (C₄F₈), perfluoropropane (C₃F₈), trifluoromethane, carbon monoxide, argon, oxygen, nitrogen, sulfur hexafluoride, difluoromethane, nitrogen trifluoride, chlorine trifluoride, chlorine, trichloroborane, and dichloroborane may be used. For the dry etching of the resist underlying film, a halogen gas is preferably used, and a fluorine-based gas is more preferably used. Examples of fluorine-based gas include tetrafluoromethane (CF₄), perfluorocyclobutane (C₄F₈), perfluoropropane (C₃F₈), trifluoromethane, and difluoromethane (CH₂F₂).

Thereafter, a part of the inorganic lower-layer film is removed using a film comprising the patterned photoresist film and the resist underlying film of the present invention as a protective film. The removal of the inorganic lower-layer film (lower layer) is preferably conducted by dry etching using a fluorine-based gas.

Finally, the semiconductor substrate is processed. The processing of the semiconductor substrate is preferably conducted by dry etching using a fluorine-based gas. Examples of fluorine-based gas include tetrafluoromethane (CF₄), perfluorocyclobutane (C₄F₈), perfluoropropane (C₃F₈), trifluoromethane, and difluoromethane (CH₂F₂).

### EXAMPLES

Hereinbelow, the present invention will be described in more detail with reference to the following examples, which should not be construed as limiting the scope of the present invention.

Identification of the compounds obtained in the Synthesis Examples below in the present specification was made by an NMR analysis. The apparatus used and the conditions for the measurement, etc., are as follows.

Apparatus: JNM-ECA500, manufactured by JEOL LTD.
Nucleus observed: Proton

### Temperature: 23° C.

### Frequency: 500 MHz

Deuterated solvent: DMSO

The weight average molecular weight values of the compounds obtained in the Synthesis Examples below in the present specification are the results of measurement by gel permeation chromatography (hereinafter abbreviated to “GPC”). The measuring apparatus and conditions for the measurement, etc., are as follows.

Apparatus: HLC-8320GPC, manufactured by Tosoh Corp.
GPC Column: Asahipak [registered trademark] GF-310HQ, Asahipak GF-510HQ, and Asahipak GF-710HQ
Column temperature: 40° C.
Flow rate: 0.6 mL/minute

### Eluent: DMF

Standard sample: Polystyrene

### Synthesis Example 1

Into a flask equipped with a stirrer, a thermometer, and a Dimroth condenser were charged 10.82 g of 1,4-phenylenediamine, 23.96 g of glutaric anhydride, and 139.02 g of tetrahydrofuran, and the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 31.27 g of a white powder (yield: 93%).

The NMR analysis confirmed formation of a compound presumed to have a structure shown below (purity: >95%). σ=1.79 (4H, quin), 2.26 (4H, t), 2.31 (4H, t), 7.48 (4H, D), 9.80 (2H, s), 12.06 (2H, br)

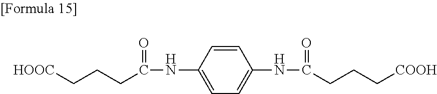 [SMILES: O=C(O)CCCC(=O)Nc1ccc(NC(=O)CCCC(=O)O)cc1]

To a flask equipped with a stirrer, a thermometer, and a Dimroth condenser were added 47.72 g of cyclohexanone (hereinafter abbreviated to “CYH” in the present specification), 11.93 g of N,N-2-trimethylpropionamide (hereinafter abbreviated to “DMIB” in the present specification), 6.60 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 16,900, as determined using a conversion calibration curve obtained from the standard polystyrene. The obtained reaction product is presumed to be a copolymer having a structural unit represented by the following formula.

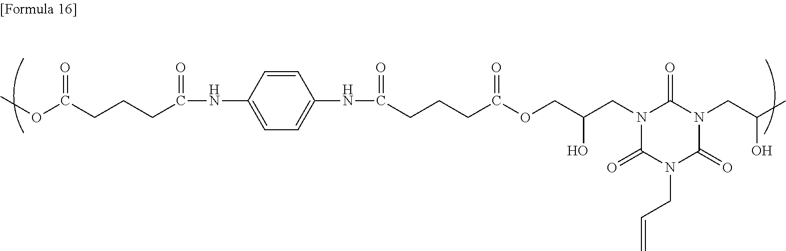

### Synthesis Example 2

Into a flask equipped with a stirrer, a thermometer, and a Dimroth condenser were charged 10.81 g of 1,3-phenylenediamine, 23.97 g of glutaric anhydride, and 139.32 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 22.69 g of a white powder (yield: 71%).

The NMR analysis confirmed formation of a compound presumed to have a structure shown below (purity: >95%). σ=1.80 (4H, quin), 2.27 (4H, t), 2.33 (4H, t), 7.17 (1H, t), 7.25 (2H, d), 7.92 (1H, s), 9.88 (2H, s), 12.07 (2H, br)

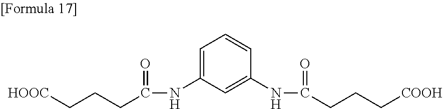 [SMILES: O=C(O)CCCC(=O)Nc1cccc(NC(=O)CCCC(=O)O)c1]

To a flask equipped with a stirrer, a thermometer, and a Dimroth condenser were added 47.68 g of CYH, 6.60 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 9,300, as determined using a conversion calibration curve obtained from the standard polystyrene. The obtained reaction product is presumed to be a copolymer having a structural unit represented by the following formula.

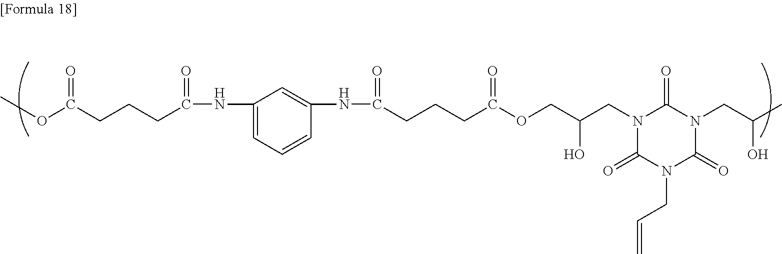

### Synthesis Example 3

Into a flask equipped with a stirrer, a thermometer, and a Dimroth condenser were charged 10.81 g of 1,2-phenylenediamine, 23.97 g of glutaric anhydride, and 140.00 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 31.25 g of a white powder (yield: 95%).

The NMR analysis confirmed formation of a compound presumed to have a structure shown below (purity: >95%). σ=1.82 (4H, quin), 2.29 (4H, t), 2.38 (4H, t), 7.12 (2H, t), 7.51 (2H, d), 9.27 (2H, s), 12.08 (2H, br)

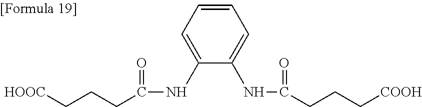 [SMILES: O=C(O)CCCC(=O)Nc1ccccc1NC(=O)CCCC(=O)O]

To a flask equipped with a stirrer, a thermometer, and a Dimroth condenser were added 47.97 g of CYH, 6.60 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 5,000, as determined using a conversion calibration curve obtained from the standard polystyrene. The obtained reaction product is presumed to be a copolymer having a structural unit represented by the following formula.

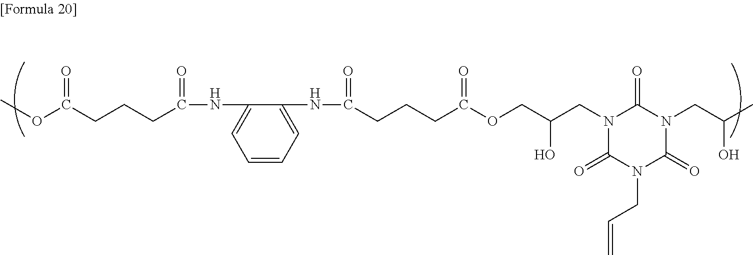

### Synthesis Example 4

Into a flask equipped with a stirrer, a thermometer, and a Dimroth condenser were charged 12.22 g of 2,4-diaminotoluene, 23.97 g of glutaric anhydride, and 146.70 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 31.25 g of a white powder (yield: 93%).

The NMR analysis confirmed formation of a compound presumed to have a structure shown below (purity: >95%). σ=1.80 (4H, quin), 2.11 (3H, s), 2.31 (8H, m), 7.07 (1H, d), 7.34 (1H, d), 7.62 (1H, d), 9.24 (1H, s), 9.82 (1H, s), 12.07 (2H, br)

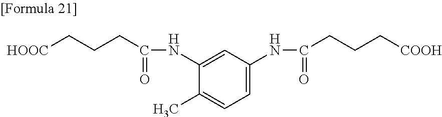 [SMILES: Cc1ccc(NC(=O)CCCC(=O)O)cc1NC(=O)CCCC(=O)O]

To a flask equipped with a stirrer, a thermometer, and a Dimroth condenser were added 49.00 g of CYH, 6.87 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 8,100, as determined using a conversion calibration curve obtained from the standard polystyrene. The obtained reaction product is presumed to be a copolymer having a structural unit represented by the following formula.

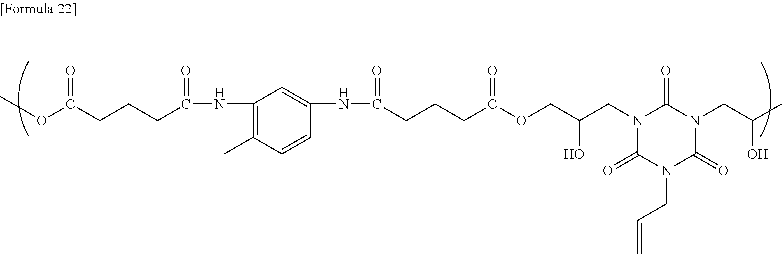

### Synthesis Example 5

Into a flask equipped with a stirrer, a thermometer, and a Dimroth condenser were charged 10.27 g of 1,3-phenylenediamine, 24.26 g of glycolic anhydride, and 138.13 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 29.83 g of a white powder (yield: 92%).

The NMR analysis confirmed formation of a compound presumed to have a structure shown below (purity: >95%). σ=4.16 (4H, s), 4.20 (4H, s), 7.24 (1H, t), 7.35 (2H, d), 7.99 (1H, s), 9.90 (2H, s), 12.86 (2H, br)

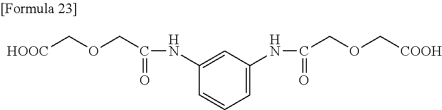 [SMILES: O=C(O)COCC(=O)Nc1cccc(NC(=O)COCC(=O)O)c1]

To a flask equipped with a stirrer, a thermometer, and a Dimroth condenser were added 48.22 g of CYH, 6.68 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 4,200, as determined using a conversion calibration curve obtained from the standard polystyrene. The obtained reaction product is presumed to be a copolymer having a structural unit represented by the following formula.

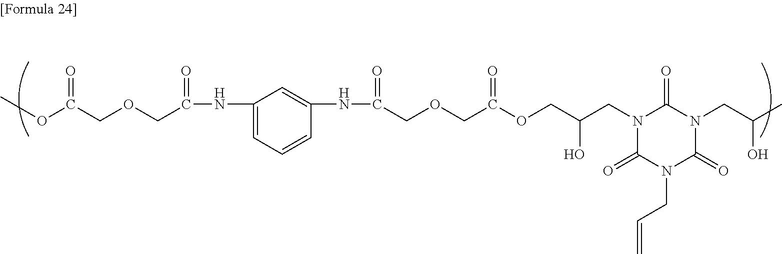

### Synthesis Example 6

Into a flask equipped with a stirrer, a thermometer, and a Dimroth condenser were charged 11.64 g of methyl 3,4-diaminobenzoate, 16.78 g of glutaric anhydride, and 114.00 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 16.32 g of a white powder (yield: 59%).

The NMR analysis confirmed formation of a compound presumed to have a structure shown below (purity: >95%). σ=1.83 (4H, quin), 2.30 (2H, t), 2.42 (2H, t), 3.83 (3H, s), 7.71 (1H, d), 7.83 (1H, d), 8.13 (1H, s), 9.42 (2H, s), 12.10 (2H, br)

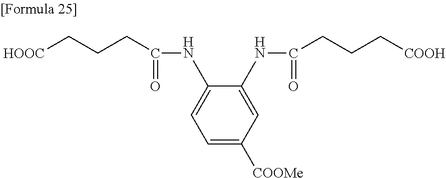 [SMILES: COC(=O)c1ccc(NC(=O)CCCC(=O)O)c(NC(=O)CCCC(=O)O)c1]

To a flask equipped with a stirrer, a thermometer, and a Dimroth condenser were added 47.68 g of CYH, 6.60 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 9,300, as determined using a conversion calibration curve obtained from the standard polystyrene. The obtained reaction product is presumed to be a copolymer having a structural unit represented by the following formula.

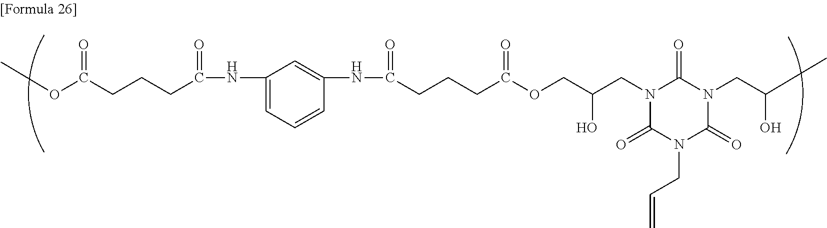

### Synthesis Example 7

Into a flask equipped with a stirrer, a thermometer, and a Dimroth condenser were charged 11.70 g of 3-amino-4-methoxybenzoic acid, 8.40 g of glutaric anhydride, and 79.60 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 15.63 g of a pale gray powder (yield: 79%).

The NMR analysis confirmed formation of a compound presumed to have a structure shown below (purity: >95%). σ=1.76 (2H, quin), 2.27 (2H, t), 2.43 (2H, t), 3.89 (3H, s), 7.12 (1H, d), 7.68 (1H, d), 8.55 (1H, s), 9.17 (1H, s), 12.35 (2H, br)

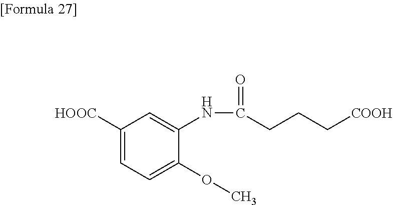 [SMILES: COc1ccc(C(=O)O)cc1NC(=O)CCCC(=O)O]

To a flask equipped with a stirrer, a thermometer, and a Dimroth condenser were added 43.33 g of CYH, 6.07 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 6,900, as determined using a conversion calibration curve obtained from the standard polystyrene. The obtained reaction product is presumed to be a copolymer having a structural unit represented by the following formula.

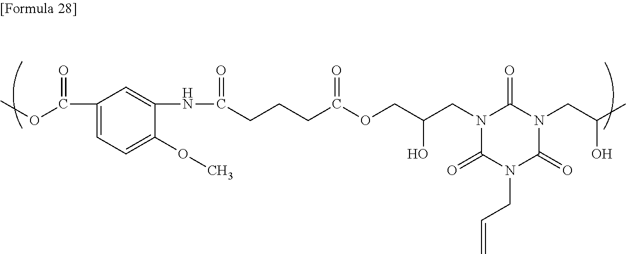

### Synthesis Example 8

Into a flask equipped with a stirrer, a thermometer, and a Dimroth condenser were charged 10.00 g of 1-methyl-2-aminoterephthalate, 6.14 g of glutaric anhydride, and 40.69 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 12.57 g of a pale yellow powder (yield: 79.3%).

The NMR analysis confirmed formation of a compound presumed to have a structure shown below (purity: >95%).: σ=1.78 (2H, quin), 2.26 (2H, t), 2.43 (2H, t), 3.89 (3H, s), 7.14 (1H, d), 7.68 (1H, d), 8.55 (1H, s), 9.17 (1H, s), 12.35 (2H, br)

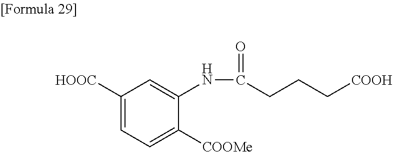 [SMILES: COC(=O)c1ccc(C(=O)O)cc1NC(=O)CCCC(=O)O]

To a flask equipped with a stirrer, a thermometer, and a Dimroth condenser were added 45.66 g of CYH, 6.07 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 7,800, as determined using a conversion calibration curve obtained from the standard polystyrene. The obtained reaction product is presumed to be a copolymer having a structural unit represented by the following formula.

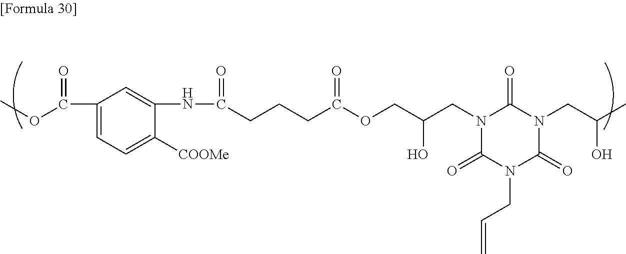

### Synthesis Example 9

Into a flask equipped with a stirrer, a thermometer, and a Dimroth condenser were charged 10.00 g of 2-amino-4-(trifluoromethyl)benzoic acid, 5.84 g of glutaric anhydride, and 39.20 g of tetrahydrofuran, and the resultant mixture was heated under reflux in a nitrogen gas atmosphere for 12 hours. After completion of the reaction, the interior of the reaction system was cooled to room temperature, and then the resultant reaction solution was poured into 300 ml of acetonitrile to allow the intended product to precipitate. The resultant precipitate was collected by filtration using a Kiriyama funnel, washed with acetonitrile, and then dried under a reduced pressure at 40° C. for 12 hours to obtain 10.54 g of a pale yellow powder (yield: 68%). The NMR analysis confirmed formation of a compound presumed to have a structure shown below (purity: >95%).: σ=1.80 (2H, quin), 2.27 (2H, t), 2.46 (2H, t), 7.44 (1H, d), 8.10 (1H, d), 8.79 (1H, s), 11.13 (1H, s), 12.30 (2H, br)

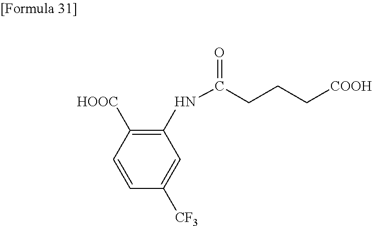 [SMILES: O=C(O)CCCC(=O)Nc1cc(C(F)(F)F)ccc1C(=O)O]

To a flask equipped with a stirrer, a thermometer, and a Dimroth condenser were added 46.54 g of CYH, 6.26 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 3,500, as determined using a conversion calibration curve obtained from the standard polystyrene. The obtained reaction product is presumed to be a copolymer having a structural unit represented by the following formula.

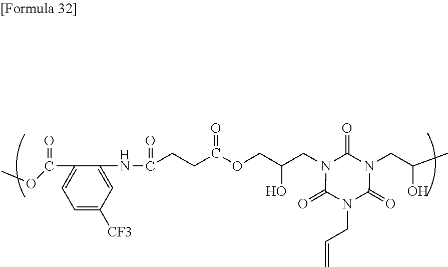

### Synthesis Example 10

Into a flask equipped with a stirrer, a thermometer, and a Dimroth condenser were charged 13.72 g of 4-aminobenzoic acid, 11.98 g of glutaric anhydride, and 102.80 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 16.55 g of a white powder (yield: 66%).

The NMR analysis confirmed formation of a compound presumed to have a structure shown below (purity: >95%).: σ=1.78 (2H, quin), 2.24 (2H, t), 2.36 (2H, t), 7.67 (2H, d), 7.83 (2H, d), 10.16 (1H, s), 12.33 (2H, br)

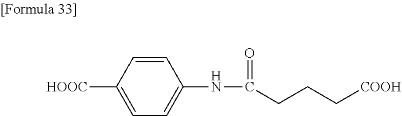 [SMILES: O=C(O)CCCC(=O)Nc1ccc(C(=O)O)cc1]

To a flask equipped with a stirrer, a thermometer, and a Dimroth condenser were added 41.15 g of CYH, 4.93 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 9,900, as determined using a conversion calibration curve obtained from the standard polystyrene. The obtained reaction product is presumed to be a copolymer having a structural unit represented by the following formula.

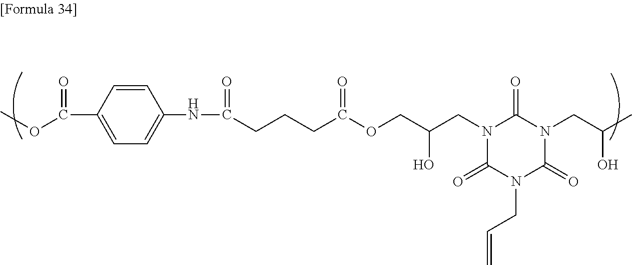

### Synthesis Example 11

Into a flask equipped with a stirrer, a thermometer, and a Dimroth condenser were charged 12.40 g of bis(4-aminophenyl) sulfone, 11.99 g of glutaric anhydride, and 97.61 g of tetrahydrofuran, and the resultant mixture was heated under reflux in a nitrogen gas atmosphere for 2 hours. After completion of the reaction, the interior of the reaction system was cooled to room temperature, and then the resultant reaction solution was poured into 300 ml of acetone to allow the intended product to precipitate. The resultant precipitate was collected by filtration using a Kiriyama funnel, washed with acetone, and then dried under a reduced pressure at 40° C. for 12 hours to obtain 22.22 g of a white powder (yield: 93%).

The NMR analysis confirmed formation of a compound presumed to have a structure shown below (purity: >95%).: σ=1.76 (4H, quin), 2.26 (4H, t), 2.35 (4H, t), 7.74 (4H, d), 7.81 (4H, d), 10.29 (2H, s), 12.04 (2H, br)

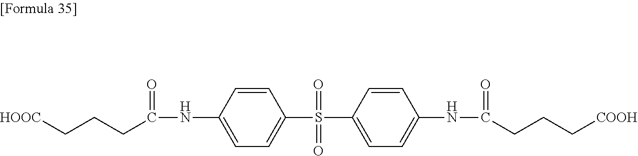 [SMILES: O=C(O)CCCC(=O)Nc1ccc(S(=O)(=O)c2ccc(NC(=O)CCCC(=O)O)cc2)cc1]

To a flask equipped with a stirrer, a thermometer, and a Dimroth condenser were added 58.72 g of CYH, 9.35 g of the obtained compound, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 13,600, as determined using a conversion calibration curve obtained from the standard polystyrene. The obtained reaction product is presumed to be a copolymer having a structural unit represented by the following formula.

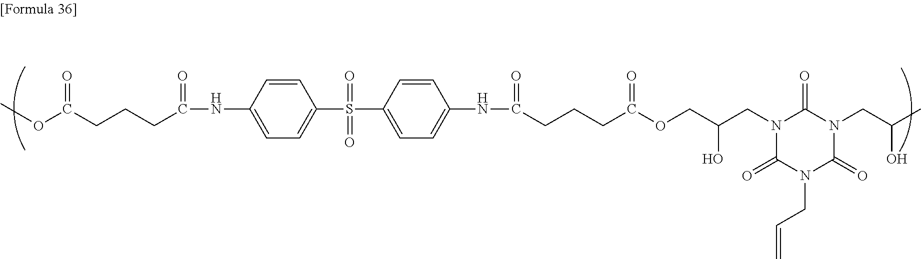

### Comparative Synthesis Example 1

To a flask equipped with a stirrer, a thermometer, and a Dimroth condenser were added 34.36 g of CYH, 3.25 g of isophthalic acid, 5.00 g of monoallyldiglycidyl isocyanurate, and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 14,700, as determined using a conversion calibration curve obtained from the standard polystyrene. The obtained reaction product is presumed to be a copolymer having a structural unit represented by the following formula.

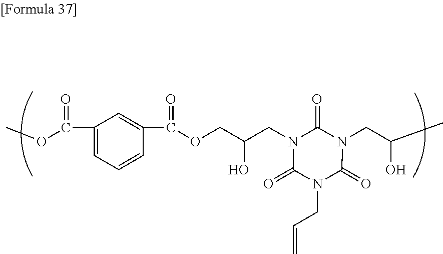

### Comparative Synthesis Example 2

To a flask equipped with a stirrer, a thermometer, and a Dimroth condenser were added 38.29 g of CYH, 3.30 g of monoallyl isocyanurate, 5.00 g of HP-4032D (manufactured by DIC Corporation), and 0.33 g of ethyltriphenylphosphonium bromide as a catalyst, and then the resultant mixture was allowed to react at 120° C. for 24 hours to obtain a solution containing the reaction product. The GPC analysis of the obtained reaction product showed that the reaction product had a weight average molecular weight of 14,800, as determined using a conversion calibration curve obtained from the standard polystyrene. The obtained reaction product is presumed to be a copolymer having a structural unit represented by the following formula.

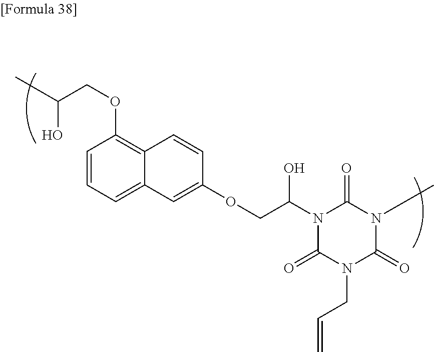

### [Preparation of Resist Underlying Film-Forming Composition]

### Example 1

Into 3.78 g of the solution obtained in Synthesis Example 1 containing 0.53 g of the copolymer (the solvent was the mixture of CYH and DMIB in a weight ratio of 8:2 used in the synthesis) were mixed 7.71 g of CYH, 1.93 g of DMIB, 0.07 g of propylene glycol monomethyl ether (hereinafter abbreviated to “PGME” in the present specification), 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 2

Into 3.12 g of the solution obtained in Synthesis Example 2 containing 0.53 g of the copolymer (the solvent was CYH used in the synthesis) were mixed 4.57 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 3

Into 3.36 g of the solution obtained in Synthesis Example 3 containing 0.53 g of the copolymer (the solvent was CYH used in the synthesis) were mixed 4.33 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 4

Into 3.01 g of the solution obtained in Synthesis Example 4 containing 0.53 g of the copolymer (the solvent was CYH used in the synthesis) were mixed 4.33 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 5

Into 2.99 g of the solution obtained in Synthesis Example 5 containing 0.53 g of the copolymer (the solvent was CYH used in the synthesis) were mixed 4.70 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 6

Into 3.26 g of the solution obtained in Synthesis Example 6 containing 0.53 g of the copolymer (the solvent was CYH used in the synthesis) were mixed 4.43 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 7

Into 3.11 g of the solution obtained in Synthesis Example 7 containing 0.53 g of the copolymer (the solvent was CYH used in the synthesis) were mixed 4.58 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 m to prepare a resist underlying film-forming composition.

### Example 8

Into 3.07 g of the solution obtained in Synthesis Example 8 containing 0.53 g of the copolymer (the solvent was CYH used in the synthesis) were mixed 4.62 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 9

Into 3.00 g of the solution obtained in Synthesis Example 9 containing 0.53 g of the copolymer (the solvent was CYH used in the synthesis) were mixed 3.38 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 10

Into 3.04 g of the solution obtained in Synthesis Example 10 containing 0.53 g of the copolymer (the solvent was CYH used in the synthesis) were mixed 3.35 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Example 11

Into 2.98 g of the solution obtained in Synthesis Example 11 containing 0.53 g of the copolymer (the solvent was CYH used in the synthesis) were mixed 3.40 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Comparative Example 1

Into 3.42 g of the solution obtained in Comparative Synthesis Example 1 containing 0.53 g of the copolymer (the solvent was CYH used in the synthesis) were mixed 3.42 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

### Comparative Example 2

Into 2.99 g of the solution obtained in Comparative Synthesis Example 2 containing 0.53 g of the copolymer (the solvent was CYH used in the synthesis) were mixed 3.39 g of CYH, 5.80 g of PGME, 0.13 g of tetramethoxymethylglycoluril (trade name: Powderlink 1174, manufactured by Nihon Cytec Industries Inc.), 1.32 g of a 1% by mass PGME solution of 5-sulfosalicylic acid (manufactured by Tokyo Chemical Industry Co., Ltd.), and 0.05 g of a 1% by mass PGME solution of a surfactant (trade name: R-30N, manufactured by DIC Corporation) to obtain a 4.5% by mass solution. The obtained solution was filtered using a polytetrafluoroethylene microfilter having a pore diameter of 0.2 μm to prepare a resist underlying film-forming composition.

[Test for Dissolution into Photoresist Solvent]

Each of the resist underlying film-forming compositions prepared in Examples 1 to 11 and Comparative Examples 1 and 2 was applied by a spinner onto a silicon wafer. Thereafter, the resultant silicon wafer was baked on a hotplate at a temperature of 215° C. for one minute to form a resist underlying film (thickness: 0.1 μm). The formed resist underlying film was immersed in PGME and propylene glycol monomethyl ether acetate, which are solvents used in the photoresist solution, to confirm that the film was insoluble in both of the solvents. Separately, the formed resist underlying film was immersed in an alkaline developer for photoresist development (2.38% by mass aqueous solution of tetramethylammonium hydroxide) to confirm that the film was insoluble in the developer. “0” indicates that the film was insoluble, and “x” indicates that the film was dissolved. The results are shown in Table 1.

[Test for Optical Parameters]

Each of the resist underlying film-forming compositions prepared in Examples 1 to 11 and Comparative Examples 1 and 2 was applied by a spinner onto a silicon wafer. Thereafter, the resultant silicon wafer was baked on a hotplate at a temperature of 215° C. for one minute to form a resist underlying film (thickness: 0.1 μm). The formed resist underlying film was subjected to determination of the refractive index (n value) at a wavelength of 248 nm and the attenuation coefficient (k value) using a spectroscopic ellipsometer (VUV-VASE VU-302, manufactured by J. A. Woollam Co., Inc.). The results are shown in Table 1 below. It is desirable for the resist underlying film to have a k value at 248 nm of 0.1 or more in order to achieve satisfactory antireflection ability.

[Measurement of Dry Etching Rate]

Using each of the resist underlying film-forming compositions prepared in Examples 1 to 11 and Comparative Examples 1 and 2, a resist underlying film was formed on a silicon wafer in the same manner as described above. Then, the dry etching rate of the formed resist underlying film was measured using RIE System, manufactured by Samco Inc., under the conditions using N₂ as a dry etching gas. Separately, a photoresist solution (trade name: V146G, manufactured by JSR Corporation) was applied by a spinner onto a silicon wafer, and baked on a hotplate at a temperature of 110° C. for one minute to form a photoresist film. The dry etching rate of the thus formed photoresist film was measured using the above-mentioned RIE System, manufactured by Samco Inc., under the conditions using N₂ as a dry etching gas. The “selective ratio” of the dry etching rate of each of the resist underlying films when taken the dry etching rate of the photoresist film as 1.00 was calculated. For a good processing using dry etching, the selective ratio is desirably 1.5 or more. The results are shown in Table 1 below.

[Evaluation of Photoresist Pattern Form]

Each of the resist underlying film-forming compositions prepared in Examples 1 to 11 and Comparative Examples 1 and 2 was applied by a spinner onto a silicon wafer. Then, the resultant silicon wafer was baked on a hotplate at 215° C. for one minute to form a resist underlying film having a thickness of 0.1 μm. A commercially available photoresist solution (trade name: SEPR-430, manufactured by Shin-Etsu Chemical Co., Ltd.) was applied by a spinner onto the resist underlying film, and baked on a hotplate at 100° C. for 60 seconds to form a photoresist film (thickness: 0.55 μm).

Then, using a scanner, NSRS205C, manufactured by Nikon Corporation (wavelength: 248 nm; NA: 0.75; σ: 0.43/0.85 (ANNULAR)), the formed photoresist film was subjected to exposure through a photomask, which was set so as to form nine (9) lines wherein the line width of the photoresist after development and the width between the lines in the photoresist were 0.17 μm, i.e., 0.17 μm L/S (dense line). Thereafter, the resultant film was subjected to post exposure bake (PEB) on a hotplate at 110° C. for 60 seconds, cooled, and then subjected to development using a 0.26 N aqueous solution of tetramethylammonium hydroxide as a developer by a 60-second single puddle step according to the industrial standards. The cross-section of the obtained photoresist pattern, when taken along the direction perpendicular to the substrate, i.e., the silicon wafer, was examined by means of a scanning electron microscope (SEM). “◯” indicates a sample, in which a photoresist was formed on the substrate and it had a good straight bottom form, and “x” indicates a sample not meeting the above. The results are shown in Table 1.

TABLE 1

| | | | | Optical parameter | | Etching | |
|---|---|---|---|---|---|---|---|
| | Solvent resistance | | | 248 nm | | selective | Lithography |
| | PGME | PGMEA | NMD-3 | n value | k value | ratio | properties |
| Example 1 | ◯ | ◯ | ◯ | 1.64 | 0.12 | 1.85 | ◯ |
| Example 2 | ◯ | ◯ | ◯ | 1.73 | 0.15 | 1.81 | ◯ |
| Example 3 | ◯ | ◯ | ◯ | 1.72 | 0.10 | 1.75 | ◯ |
| Example 4 | ◯ | ◯ | ◯ | 1.72 | 0.11 | 1.75 | ◯ |
| Example 5 | ◯ | ◯ | ◯ | 1.73 | 0.16 | 2.07 | ◯ |
| Example 6 | ◯ | ◯ | ◯ | 1.68 | 0.10 | 1.82 | ◯ |
| Example 7 | ◯ | ◯ | ◯ | 1.71 | 0.14 | 1.87 | ◯ |
| Example 8 | ◯ | ◯ | ◯ | 1.72 | 0.13 | 1.87 | ◯ |
| Example 9 | ◯ | ◯ | ◯ | 1.65 | 0.11 | 1.67 | ◯ |
| Example10 | ◯ | ◯ | ◯ | 1.57 | 0.10 | 1.81 | ◯ |
| Example11 | ◯ | ◯ | ◯ | 1.57 | 0.13 | 1.78 | ◯ |
| Comparative | ◯ | ◯ | ◯ | 1.82 | 0.04 | 1.71 | X |
| Example 1 | | | | | | | |
| Comparative | ◯ | ◯ | ◯ | 1.85 | 0.33 | 1.35 | ◯ |
| Example 2 | | | | | | | |

As can be seen from the results shown in Table 1 above, the resist underlying films formed from the resist underlying film-forming compositions prepared in Examples 1 to 11 exhibit a k value larger than 0.1 at 248 nm, which indicates that they have satisfactory antireflection ability in a KrF process. In contrast, the resist underlying film formed from the resist underlying film-forming composition prepared in Comparative Example 1 exhibits a k value less than 0.1, which shows that it has insufficient antireflection ability. Moreover, the resist underlying films formed from the resist underlying film-forming compositions prepared in Examples 1 to 11 have a selective ratio remarkably larger than 1.5 relative to the dry etching rate of the photoresist film, which indicates that they have satisfactory dry etching rate. In contrast, the resist underlying film formed from the resist underlying film-forming composition prepared in Comparative Example 2 has a selective ratio as small as 1.35, which shows that it has a low dry etching rate. Furthermore, the cross-section profile of the photoresist pattern obtained using each of the resist underlying film-forming compositions prepared in Examples 1 to 11 had a good straight bottom form. In contrast, when the resist underlying film-forming composition prepared in Comparative Example 1 was used, disappearance of the pattern after the development was observed.

The above results demonstrated that the resist underlying film-forming compositions prepared in Examples 1 to 11 could provide a resist underlying film having a high dry etching rate and an antireflection ability in a KrF process.

### INDUSTRIAL APPLICABILITY

According to the present invention, there is provided a resist underlying film-forming composition, which provides a resist underlying film exhibiting, particularly in a KrF process, a satisfactory antireflection ability, high solvent resistance and high dry etching rate, and permits formation of a photoresist pattern with a good cross-section profile.

1-11. (canceled)

12. A compound represented by the following formula (1): 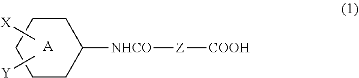
wherein:
A represents a benzene ring or a cyclohexane ring,
X represents a hydrogen atom, an optionally halo-substituted alkyl or alkoxy group having 1 to 10 carbon atoms, or an alkoxycarbonyl group having 2 to 11 carbon atoms,
Y represents —COOH or -L-NHCO—Z—COOH,
Z represents an alkylene group having 3 to 10 carbon atoms and being optionally interrupted by an oxygen atom, a sulfur atom, or a nitrogen atom, and
L represents a single bond or a spacer group,
with the proviso that the following compounds are excluded:

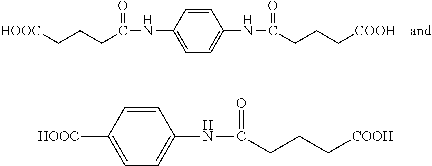 [SMILES: O=C(O)CCCC(=O)Nc1ccc(NC(=O)CCCC(=O)O)cc1 ; O=C(O)CCCC(=O)Nc1ccc(C(=O)O)cc1]

13. The compound according to claim 12, wherein A is a benzene ring.

14. The compound according to claim 12, wherein the spacer group -L- is represented by the following formula: 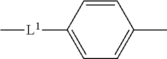
wherein L¹ represents a single bond, an oxygen atom, a carbonyl group, a sulfonyl group, or an optionally halo-substituted alkylene group having 1 to 6 carbon atoms.

15. The compound according to claim 13, wherein the spacer group -L- is represented by the following formula: 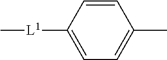
wherein L¹ represents a single bond, an oxygen atom, a carbonyl group, a sulfonyl group, or an optionally halo-substituted alkylene group having 1 to 6 carbon atoms.

16. A copolymer comprising structural unit (A) derived from a diepoxy compound, and structural unit (B) derived from a compound represented by the following formula (1): 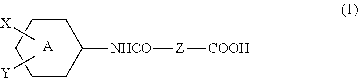
wherein:
A represents a benzene ring or a cyclohexane ring,
X represents a hydrogen atom, an optionally halo-substituted alkyl or alkoxy group having 1 to 10 carbon atoms, or an alkoxycarbonyl group having 2 to 11 carbon atoms,
Y represents —COOH or -L-NHCO—Z—COOH,
Z represents an alkylene group having 3 to 10 carbon atoms and being optionally interrupted by an oxygen atom, a sulfur atom, or a nitrogen atom, and
L represents a single bond or a spacer group.

17. The copolymer according to claim 16, wherein the diepoxy compound is represented by the following formula (2): 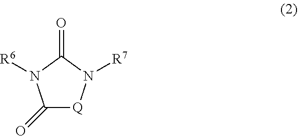
wherein:
R⁶ and R⁷ represent an epoxy-containing group, which may be the same or different, and
Q represents a group represented by the following formula (31), (32), or (33): 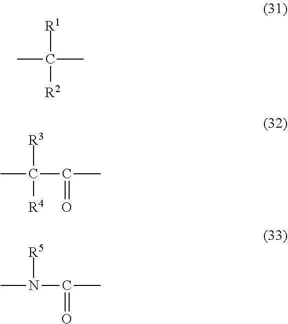
wherein each of R¹ to R⁴ independently represents a hydrogen atom, an alkyl group having 1 to 6 carbon atoms, an alkenyl group having 3 to 6 carbon atoms, a benzyl group, or a phenyl group, wherein the phenyl group is optionally substituted with at least one group selected from the group consisting of an alkyl group having 1 to 6 carbon atoms, a halogen atom, an alkoxy group having 1 to 6 carbon atoms, a nitro group, a cyano group, and an alkylthio group having 1 to 6 carbon atoms, and wherein R¹ and R², or R³ and R⁴ are optionally bonded to each other to form a ring having 3 to 6 carbon atoms, and
R⁵ represents a hydrogen atom, an alkyl group having 1 to 6 carbon atoms, an alkenyl group having 3 to 6 carbon atoms, an ether oxygen-interrupted alkyl group having 3 to 8 carbon atoms, a benzyl group, or a phenyl group.

18. The copolymer according to claim 16, wherein the diepoxy compound is represented by the following formula (4): 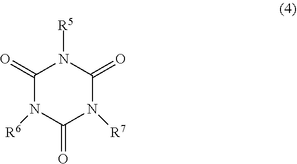
wherein:
R⁵ represents a hydrogen atom, an alkyl group having 1 to 6 carbon atoms, an alkenyl group having 3 to 6 carbon atoms, an ether oxygen-interrupted alkyl group having 3 to 8 carbon atoms, a benzyl group, or a phenyl group, and
R⁶ and R⁷ represent an epoxy-containing group.

19. A resist underlying film-forming composition comprising a solvent and the copolymer according to claim 16.

20. A resist underlying film-forming composition comprising a solvent and the copolymer according to claim 17.

21. A resist underlying film-forming composition comprising a solvent and the copolymer according to claim 18.

22. A resist underlying film-forming composition comprising a solvent and the copolymer of claim 16, the composition giving a resist underlying film which absorbs a light having a wavelength of 248 nm.

23. A resist underlying film-forming composition comprising a solvent and the copolymer of claim 17, the composition giving a resist underlying film which absorbs a light having a wavelength of 248 nm.

24. A resist underlying film-forming composition comprising a solvent and the copolymer claim 18, the composition giving a resist underlying film which absorbs a light having a wavelength of 248 nm.

25. A method for forming a resist pattern for use in producing a semiconductor device, comprising the steps of: applying the resist underlying film-forming composition according to claim 19 onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; and subjecting the exposed resist film to development.

26. A method for forming a resist pattern for use in producing a semiconductor device, comprising the steps of: applying the resist underlying film-forming composition according to claim 22 onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; and subjecting the exposed resist film to development.

27. A resist underlying film comprising the copolymer according to claim 16, wherein the resist underlying film absorbs a light having a wavelength of 248 nm.

28. A resist underlying film comprising the copolymer according to claim 17, wherein the resist underlying film absorbs a light having a wavelength of 248 nm.

29. A resist underlying film comprising the copolymer according to claim 18, wherein the resist underlying film absorbs a light having a wavelength of 248 nm.

30. A method for producing a semiconductor device, comprising the steps of: applying the resist underlying film-forming composition according to claim 19 onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; subjecting the exposed resist film to development; and processing the semiconductor substrate using the resist film as a mask.

31. A method for producing a semiconductor device, comprising the steps of: applying the resist underlying film-forming composition according to claim 22 onto a semiconductor substrate and baking the applied composition to form a resist underlying film; applying a resist onto the resist underlying film and baking the applied resist to form a resist film; subjecting the semiconductor substrate covered with the resist underlying film and the resist film to exposure to a light having a wavelength of 248 nm; subjecting the exposed resist film to development; and processing the semiconductor substrate using the resist film as a mask.
